(19) United States
(12) Patent Application Publication
(10) Pub. No.: US 2025/0341489 A1
(43) Pub. Date: Nov. 6, 2025

(54) AN ELECTROCHEMICAL SENSOR AND METHOD FOR DETECTING PATHOGENIC METABOLITES

(71) Applicant: DURHAM UNIVERSITY, Durham (GB)

(72) Inventor: Ritu Kataky, Durham (GB)

(73) Assignee: DURHAM UNIVERSITY, Durham (GB)

(21) Appl. No.: 18/867,752

(22) PCT Filed: Apr. 27, 2023
(86) PCT No.: PCT/GB2023/051124

§ 371 Date: Nov. 20, 2024

(30) Foreign Application Priority Data
May 26, 2022 (GB) 2207813.3

(51) Int. Cl. G01N 27/327; G01N 27/30; G01N 33/487

(52) U.S. Cl. CPC G01N 27/3277; G01N 27/301; G01N 33/48721; G01N 2400/18; G01N 2469/00

(57) ABSTRACT

An electrochemical sensor and a method for detecting pathogenic metabolites such as viral or bacterial metabolites are presented. The electrochemical sensor includes a first electrode modified with oligosaccharide molecules. In the detection method, a first electrode modified with oligosaccharide molecules is provided and a sample is applied on the first electrode. An electrochemical response is then measured using the first electrode to detect pathogenic metabolites in the sample.

### TECHNICAL FIELD

The present disclosure relates to an electrochemical sensor. In particular, the present disclosure relates to an electrochemical sensor and corresponding method for detecting pathogenic metabolites such as bacterial or viral metabolites.

### BACKGROUND

Electrochemical sensing may be used as relatively simple technique for pathogen identification via the detection of redox-active metabolites on an electrode surface. Bacteria can communicate using a mechanism known a quorum sensing (QS) via the secretion of signaling molecules and autoinducers to detect variations in concentration of signaling molecules. Quorum sensing (QS) allows various processes to be controlled including among others biofilm formation and the production of secondary metabolites. In previous work, Bukleman and co-workers demonstrated that QS regulated virulence factor production, can be analysed electrochemically for the accurate and sensitive evaluation of QS activation and inhibition in wild-type bacteria (Ohad Bukelman et al, “Electrochemical analysis of quorum sensing inhibition,” Chem. Commun., p. 2836-2838, 2009). Buzid and co-workers have reported the use of unmodified Boron Doped Diamond electrodes (BDDE) without modification, for simultaneous determination of pyocyanin (PYO), 2-heptyl-3-hydroxy-4-quinolone (PQS) and 2-heptyl-4-hydroxyquinoline (HHQ) in a mixed solution to analyse supernatant extracts from P. aeruginosa wild-type strains (Alyah Buzid, et al, “Molecular Signature of Pseudomonas aeruginosa with Simultaneous Nanomolar Detection of Quorum Sensing Signaling Molecules at a Boron-Doped Diamond Electrode,” Scientific Reports, vol. 6, p. 30001, 2016). This work was reported as an improvement in limits of detection reported in previous work using BDDE thin film electrodes where only PQS was measured (Matthew P Fletcher, et al “Biosensor-based assays for PQS, HHQ and related 2-alkyl-4-quinolone quorum sensing signal molecules,” Nature protocols, vol. 2, pp. 1254-62, 2077). Other reports include the use of biosensing assays (Fengjun Shang, et al, “Selective detection of dopamine using a combined permselective film of electropolymerized (poly-tyramine and poly-pyrrole-1-propionic acid) on a boron-doped diamondelectrode,” Analyst, vol. 134, pp. 519-527, 2009; Matthew P Fletcher, et al, “A dual biosensor for 2-alkyl-4-quinolone quorum-sensing signal molecules,” Environmental microbiology, vol. 9, pp. 2683-93, 2007).

PYO has been detected using Adsorptive stripping voltammetry (AdSV) using a hanging mercury drop electrode and differential pulse voltammetry (DPV) using, graphite rods and disposable screen-printed electrodes by square wave voltammetry (SWV) (Ohad Bukelman et al, “Electrochemical analysis of quorum sensing inhibition,” Chem. Commun., p. 2836-2838, 2009; D V Vukomanovic, et al “Analysis of pyocyanin from Pseudomonas aeruginosa by adsorptive stripping voltammetry,” pharmacological and toxicological methods, vol. 36, pp. 97-102, 1996; Hunter J. Sismaet, et al, “Up-regulating pyocyanin production by amino acid addition for early electrochemical identification of Pseudomonas aeruginosa,” Analyst, vol. 139, pp. 4241-4246, 2014).

Sensors suitable for integration into bandages and nanofluidic platforms, based on electrochemical detection have also been reported (Daniel L. Bellin, et al, “Integrated circuit-based electrochemical sensor for spatially resolved detection of redox-active metabolites in biofilms,” Nature Communications, vol. 5, p. 3256, 2014; Duncan Sharp, et al, “Approaching intelligent infection diagnostics: Carbon fibre sensor for electrochemical pyocyanin detection,” Bioelectrochemistry, vol. 77, pp. 114-9., 2010).

For the detection of multiple phenazines such as PQS and PYQ, conductive polymer film modified glassy carbon electrodes and preconcentration techniques have been reported (Julie Oziat, et al, “Electrochemistry provides a simple way to monitor Pseudomonas aeruginosa metabolites,” Annual International Conference of the IEEE Engineering in Medicine and Biology Society, pp. 7522-5, 2015; T Seviour, et al, “Voltammetric profiling of redox-active metabolites expressed by Pseudomonas aeruginosa for diagnostic purposes,” Chemical communications, vol. 51, pp. 3789-92, 2015).

Bellin et al report an electrochemical camera chip capable of simultaneous spatial imaging of multiple redox-active phenazine metabolites produced by Pseudomonas aeruginosa PA14 colony biofilms (Daniel L. Bellin, et al, “Electrochemical camera chip for simultaneous imaging of multiple metabolites in biofilms,” Nature Communications, vol. 7, p. 10535, 2016). A publication by Oziat and co-workers used unmodified Glassy Carbon electrodes to differentiate between Pseudomonas aeruginosa strains and its isogenic mutants, using square wave voltammetry. They observed distinctive redox signals showing PYO and Pseudomonas Quinolone signals (Julie Oziat, et al, “Electrochemical detection of redox molecules secreted by Pseudomonas aeruginosa—Part 1: Electrochemical signatures of different strains,” Bioelectrochemistry, vol. 140, p. 107747, 2021). Existing techniques, however, have a limited specificity.

It is an object of the disclosure to address one or more of the above mentioned limitations.

### SUMMARY

According to a first aspect of the disclosure there is provided an electrochemical sensor for detecting pathogenic metabolites, wherein the electrochemical sensor comprises a first electrode modified with oligosaccharide molecules.

For instance the electrochemical sensor may be used for detecting viral or bacterial metabolites. The oligosaccharide molecules may include cyclodextrin molecules or maltodextrin molecules.

Optionally, the first electrode is modified with cyclic oligosaccharide molecules.

Optionally, the cyclic oligosaccharide molecules comprise cyclodextrins or modified cyclodextrins or a combination of both.

Optionally, the first electrode is modified with at least one of alpha-cyclodextrins, beta-cyclodextrins and gamma-cyclodextrins.

Optionally, the first electrode is modified by electro-polymerization.

Optionally, the electrochemical sensor comprises a second electrode and a third electrode, wherein the first electrode is a working electrode, the second electrode is a reference electrode, and the third electrode is a counter electrode.

Optionally, the first electrode, the second electrode and the third electrode are screen printed electrodes.

According to a second aspect of the disclosure there is provided an electrochemical system comprising an electrochemical sensor according to the first aspect of the disclosure, and a potentiostat coupled to the electrochemical sensor, wherein the potentiostat is configured to perform an electrochemical technique.

For instance the electrochemical technique may include voltammetry, amperometry or impedance.

Optionally, the electrochemical sensor and the potentiostat are integrated in a portable device or a wearable device.

The electrochemical system according to the second aspect of the disclosure may comprise any of the features described above in relation to the electrochemical sensor according to the first aspect of the disclosure.

According to a third aspect of the disclosure, there is provided a method of detecting pathogenic metabolites, the method comprising
providing a first electrode modified with oligosaccharide molecules;
applying a sample on the first electrode; and
measuring an electrochemical response using the first electrode to detect pathogenic metabolites in the sample.

For instance the sample may be fluid or a gel that may comprise pathogens. For example the sample may be a biological fluid such as blood serum or urine, or a food fluid such as water or milk or any drinkable fluid. The method may be used to detect viral or bacterial metabolites.

Optionally, the pathogenic metabolites comprise redox-active metabolites.

Optionally, the pathogenic metabolites comprise phenazine metabolites. For instance, the phenazine metabolites may be Pseudomonas phenazines.

Optionally, the phenazine metabolites comprise at least one of pyocyanin (PYO), phenazine 1 carboxylic acid (PCA), 1-hydroxyphenazine (1-OHPHZ), and phenazine-1-carboxylic acid (PCN).

Optionally, the method comprises performing an electrochemical technique to obtain the electrochemical response.

Optionally, the electrochemical technique is a stripping voltammetry technique, and the electrochemical response comprises a voltammogram.

Optionally, wherein the stripping voltammetry technique is adsorptive stripping voltammetry. For instance the stripping voltammetry technique may be square wave adsorptive stripping voltammetry (SWASV).

Optionally, the first electrode is modified with cyclic oligosaccharide molecules.

For instance the cyclic oligosaccharide molecules comprise cyclodextrin or modified cyclodextrin or a combination of both. For example the first electrode may be modified with at alpha-cyclodextrin or beta-cyclodextrin or gamma-cyclodextrin, or a combination of alpha, beta and gamma-cyclodextrin.

Optionally, the method further comprises determining an amount of pathogenic metabolites.

For instance determining an amount of pathogenic metabolites may comprise determining a concentration of metabolite using a calibration curve.

Optionally, the method further comprises deconvoluting the electrochemical response to identify one or more redox peaks associated with a specific pathogenic species.

For instance one or more redox peaks may be associated with a specific bacterial species.

According to a fourth aspect of the disclosure, there is provided a method of manufacturing an electrochemical sensor for detecting pathogenic metabolites, the method comprising providing a first electrode and modifying the first electrode with oligosaccharide molecules.

Optionally, modifying the first electrode comprises performing electro-polymerization.

For instance electro-polymerization may be performed using continuous potential cycling.

The options described with respect to the first aspect of the disclosure are also common to the second, third and fourth aspects of the disclosure.

### DESCRIPTION OF THE DRAWINGS

The disclosure is described in further detail below by way of example and with reference to the accompanying drawings, in which:

FIG. 1 is a flow chart of a method for detecting pathogenic metabolites;

FIG. 2 is a flow chart of a method for modifying an electrode with cyclodextrin;

FIG. 3 is a diagram of a set up for performing electrode activation;

FIG. 4A is a diagram showing the structure of an alpha-cyclodextrin (α-CD);

FIG. 4B is a diagram showing the structure of a beta-cyclodextrin (β-CD);

FIG. 4C is a diagram showing the structure of a gamma-cyclodextrin (γ-CD);

FIG. 5A is a measurement showing the cyclic voltammograms obtained by the β-CD electro-polymerization process on screen printed electrodes;

FIG. 5B is a schematic representation of a carbon electrode with several cyclodextrin molecules electrodeposited on its surface;

FIG. 6A is a diagram of an electrochemical sensor provided with a cyclodextrin modified working electrode, a reference electrode and a counter electrode in a circular geometry;

FIG. 6B is a diagram of an electrochemical sensor provided with a cyclodextrin modified working electrode, a reference electrode and a counter electrode in a square-like geometry;

FIG. 6C is a diagram of an electrochemical sensor provided with a cyclodextrin modified working electrode, a reference electrode and a counter electrode in another geometry;

FIG. 7A is a table listing phenazine molecules present in Pseudomonas species (Pseudomonas phenazines), along with their structure and redox potential in aqueous solution;

FIG. 7B is a cyclic voltammetry graph of β-CD/SPCE in 50 μM PYO in PBS;

FIG. 7C is a cyclic voltammetry graph of β-CD/SPCE in 50 μM 1-OHPHZ in PBS;

FIG. 7D is a cyclic voltammetry graph of β-CD/SPCE in 50 μM PCA in PBS;

FIG. 7E is a diagram illustrating the structure of prodigiosin;

FIG. 8A is a cyclic voltammetry graph obtained using a beta-cyclodextrin modified screen printed carbon electrode (β-CD/SPCE) in a ternary solution mixture of 1-OHPHZ, PCA and PYO in PBS pH 7 (solid line) and control (dotted line);

FIG. 8B is a graph showing the cyclic voltammetry measurements as in FIG. 8A obtained for different scan rates;

FIG. 8C is a cyclic voltammogram obtained before and after adding 3 μM 1-OHPHZ into PBS;

FIG. 8D is a different pulse voltammogram obtained before and after adding 3 μM 1-OHPHZ into PBS;

FIG. 8E is a square wave adsorptive stripping voltammogram (SWASV) obtained before and after adding 3 μM 1-OHPHZ into PBS;

FIG. 9 is a plot showing the square wave adsorptive stripping voltammetry SWASV measurement obtained for a ternary solution mixture of 12.5 μM 1-OHPHZ, 12.5 μM PCA and 12.5 μM PYO in PBS buffer at a β-CD/SPCE;

FIG. 10A is a table showing SWASV parameters obtained for the detection of a three phenazine compounds when using a bare electrode and a beta-cyclodextrin modified electrode, respectively;

FIG. 10B is a diagram showing SWASV voltammograms obtained on β-CD modified SPCE for different mixed of phenazine concentration in PBS buffer;

FIG. 11A is a diagram showing SWASV voltammograms obtained for different concentrations of mixed phenazines;

FIG. 11B is a calibration chart showing the calibration curves for different phenazine molecules;

FIG. 12A shows the SWASV measurement of Pseudomonas fluorescence obtained in lysogeny broth growth medium after 3 days;

FIG. 12B shows the SWASV measurement of Pseudomonas aeruginosa obtained in lysogeny broth growth medium after 3 days;

FIGS. 13A and 13B show the SWASVs measurements of Serratia marcescens in lysogeny broth growth medium after 3 days in normal and acidic conditions, respectively;

FIG. 14 is a diagram of a portable device provided with an electrochemical sensor.

### DESCRIPTION

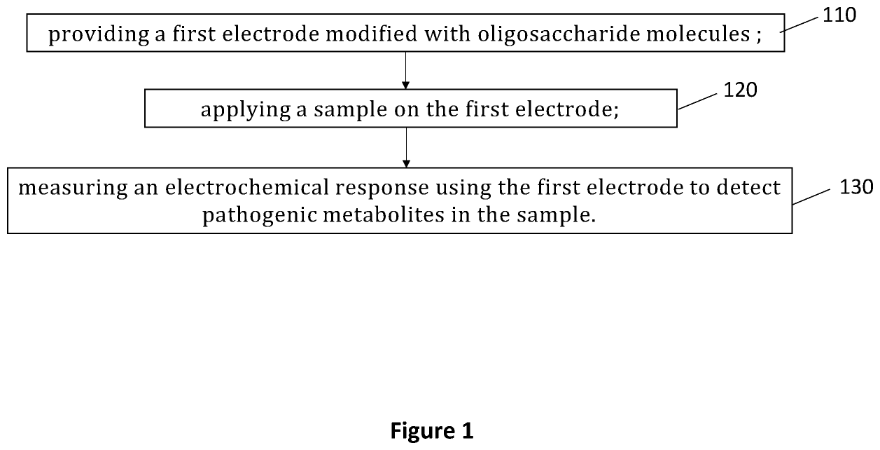

FIG. 1 is a flow chart of a method for detecting pathogenic metabolites. At step 110, a first electrode modified with oligosaccharide molecules is provided. For instance the first electrode may be modified or functionalised with cyclic oligosaccharide molecules. The cyclic oligosaccharide molecules may include cyclodextrins or modified cyclodextrins or a combination of both. For example cyclodextrins can be modified with a thiol group to form a thiolated cyclodextrins, or with a carboxyl group to form carboxyl-modified cyclodextrins etc. . . . . Other examples of cyclic oligosaccharide molecules may include maltodextrins. The type of oligosaccharide molecules used to modify the first electrode may be chosen to detect specific metabolites.

At step 120, a sample is applied on the first electrode. For instance the sample may be a fluid or a gel that may include pathogens. For example a biological fluid such as blood serum or urine, or a food fluid such as water or milk or any drinkable fluid.

At step 130, an electrochemical response is measured using the first electrode to detect pathogenic metabolites in the sample. The method may be used to detect viral or bacterial metabolites.

For instance an electrochemical technique such as a stripping voltammetry technique may be performed to obtain the electrochemical response. In turn the electrochemical response may be analyzed to determine an amount of metabolite in the sample. Different types of metabolites may also be identified based on electrochemical response. For instance, the electrochemical response may comprise a voltammogram.

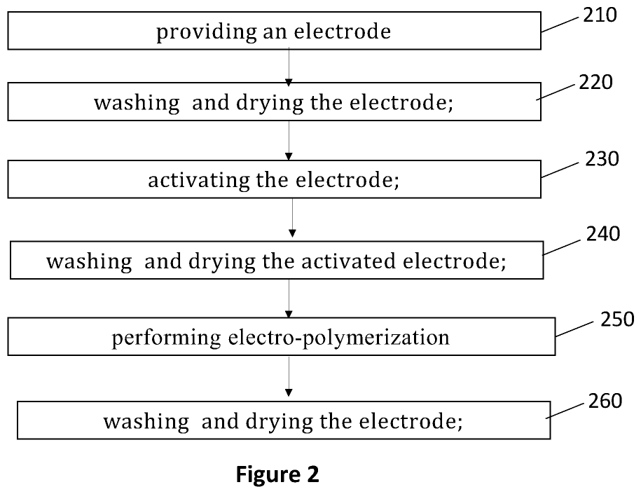

FIG. 2 a flow chart of a method for modifying an electrode with cyclodextrin. At step 210 an electrode is provided. For instance the electrode may be a printed electrode such as a screen printed electrode, or a 3D printed electrode. The electrode may be a carbon electrode.

At step 220 the electrode is washed and dried. For instance the electrode may be sonicated in acetone for several minutes (example 3 minutes), then washed with deionized water and then allowed to dry.

At step 230 the electrode is activated. Electrode activation may be performed in different fashion. For a 3D printed carbon working electrode, activation can be performed using a platinum wire counter electrode, an Ag/AgCl reference electrode in a phosphate buffer solution PBS (pH7) by applying a constant voltage of 2 V on the Ag/AgCl reference electrode for 300 s.

At step 240 the activated electrode is washed and dried. Washing can be performed with ethanol and deionized water. The activated electrode can then be allowed to dry for 24 h at room temperature.

At step 250 electro-polymerization is performed on the activated electrode. For instance, the activated printed carbon electrode may be modified using continuous potential cycling from −2 to 2 mV at a sweep rate of 20 mV/s for 10 cycles, in a solution containing 0.01M α-β-γ-Cyclodextrin in PBS pH7.

At step 260 the modified electrode is washed and dried. For instance, the modified electrode may be washed with the deionized water to remove adsorbed materials on the surface and then dried at a room temperature for further use.

In the present example a solution containing α-β-γ-Cyclodextrin has been chosen, hence allowing probing molecules of different sizes. Depending on the application a solution containing only one type of cyclodextrin may be chosen, for instance only β-Cyclodextrin.

It will also be appreciated that the method may be adapted to modify the electrode with other oligosaccharide molecules, for instance using maltodextrins.

Scanning electron microscopy (SEM) and cyclic voltammetry (CV) techniques can be used to characterize the morphology and electrical conductivity of the modified electrode.

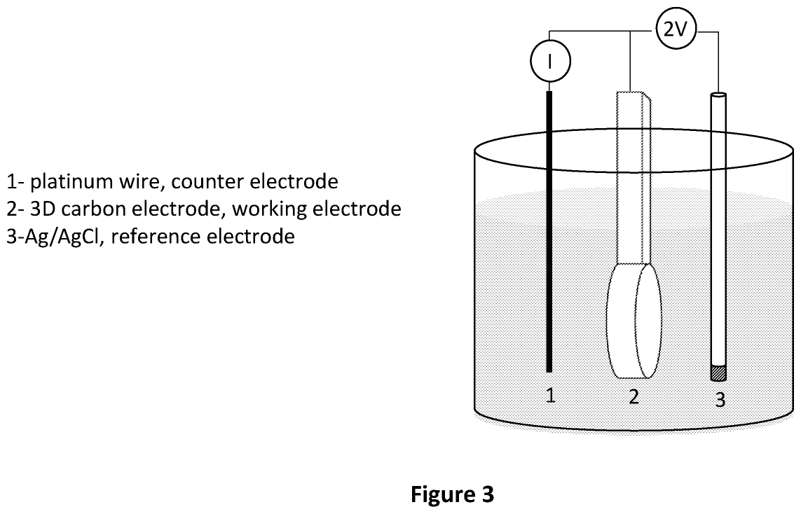

FIG. 3 is a diagram illustrating a set up for performing electrode activation.

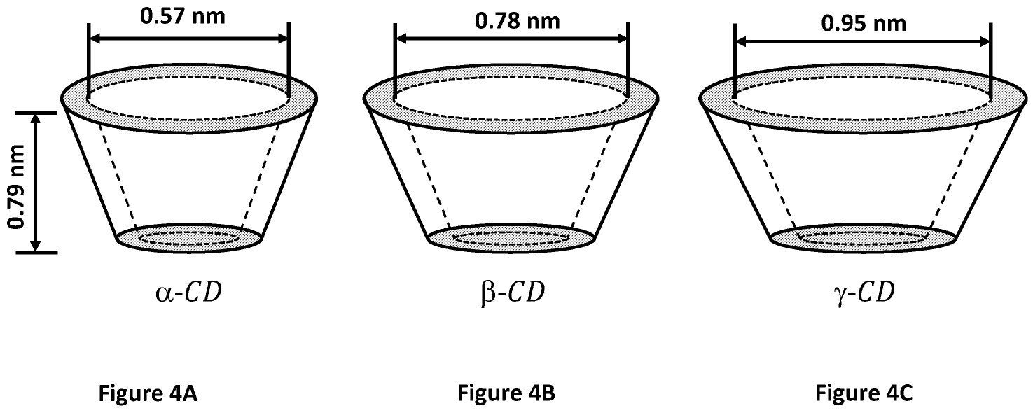

FIG. 4 illustrates the structural shape of cyclodextrin molecules. Cyclodextrins have toroidal hydrophobic cavities with a hydrophilic exterior and have been used for molecular recognition, due to their natural size and charge selective cavity (Ritu Kataky, et al “Potentiometric, enantioselective sensors for alkyland aryl ammonium ions of pharmaceutical significance, based on lipophilic cyclodextrins,” Scand J Clin Lab Inves, vol. 55, pp. 409-419, 1995; Ritu Kataky, et al, “Alkylated cyclodextrin-based potentiometric and amperometric electrodes applied to the measurement of tricyclic antidepressants,” Electroanalysis, vol. 9, pp. 1267-1272, 1997). The CD cavities can provide large, catalytic enhancement of reactions when the geometry of the substrate-CD complex is optimal.

Cyclodextrins contain several glucose monomers ranging from six to eight units in a ring, creating a cone shape. FIG. 4A shows the structure of an alpha-cyclodextrin (α-CD) containing six glucose units. FIG. 4B shows the structure of a beta-cyclodextrin (β-CD) containing seven glucose units. FIG. 4C shows the structure of a gamma-cyclodextrin (γ-CD) containing eight glucose units. The cone shape forms an open cavity which can be used to receive a target molecule. The opening of the cone shape has a diameter which increases with the number of glucose units has shown in FIGS. 4A, B and C for α-CD, β-CD, and γ-CD, respectively. The structure of the β-CD (height of 0.79±0.01 nm, exterior diameter 1.54±0.04 nm) enables the incorporation of lipophilic structures with appropriate size fit into its cavity.

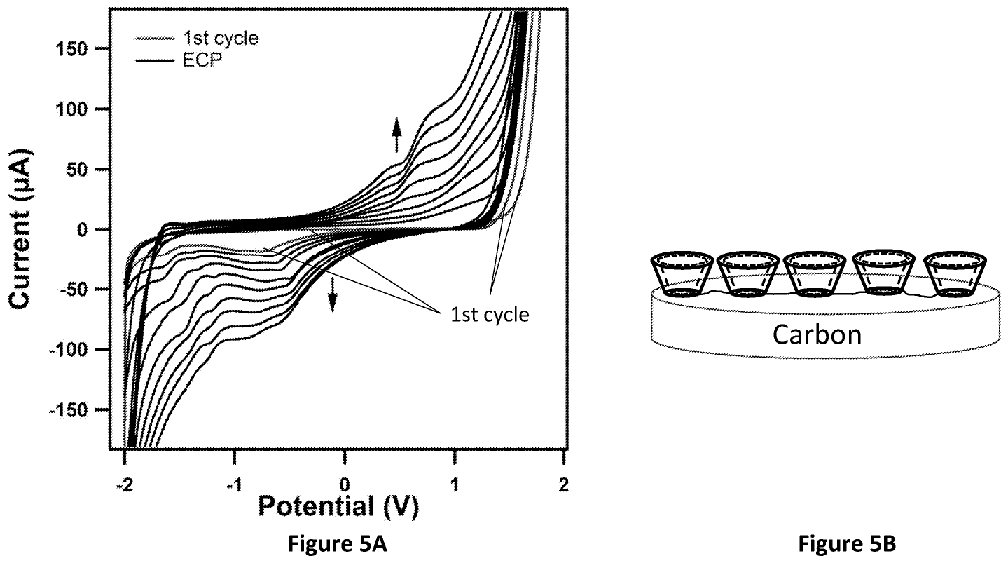

FIG. 5A shows the cyclic voltammograms obtained by the β-CD electro-polymerization process on screen printed electrode. Electrochemical measurements were done using Gamry PE-1000 potentiostat and for polymerization step 3D/screen printed carbon electrode, platinum wire and Ag/AgCl electrode were used as the working, counter and refence electrodes, respectively.

FIG. 5B is a diagram illustrating a carbon electrode with several cyclodextrin molecules electrodeposited on its surface. The modified electrode described above may be used to detect various pathogenic metabolites including bacterial metabolites such as phenazine metabolites. The type of cyclodextrin α-CD, β-CD or γ-CD, may be selected to detect a metabolite having a specific size or within a specific range.

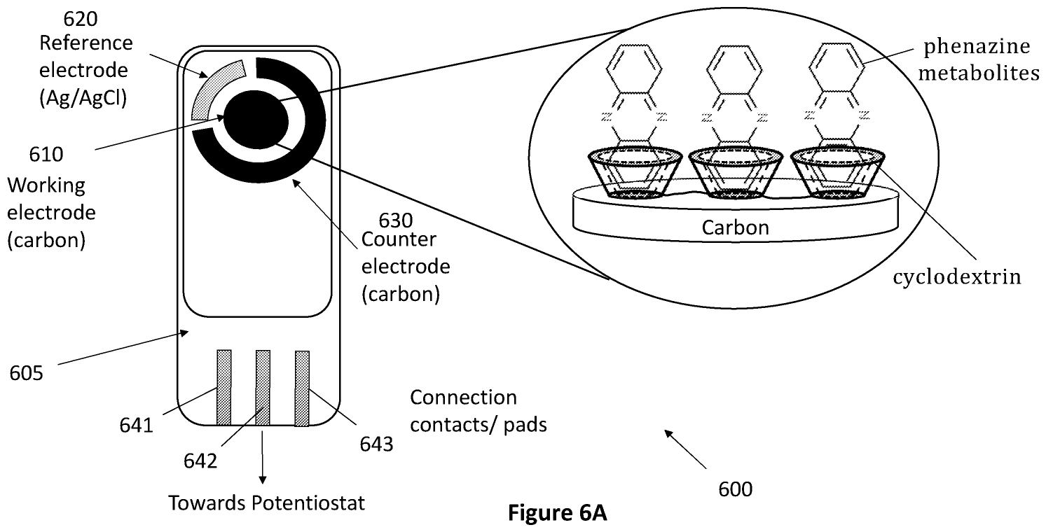

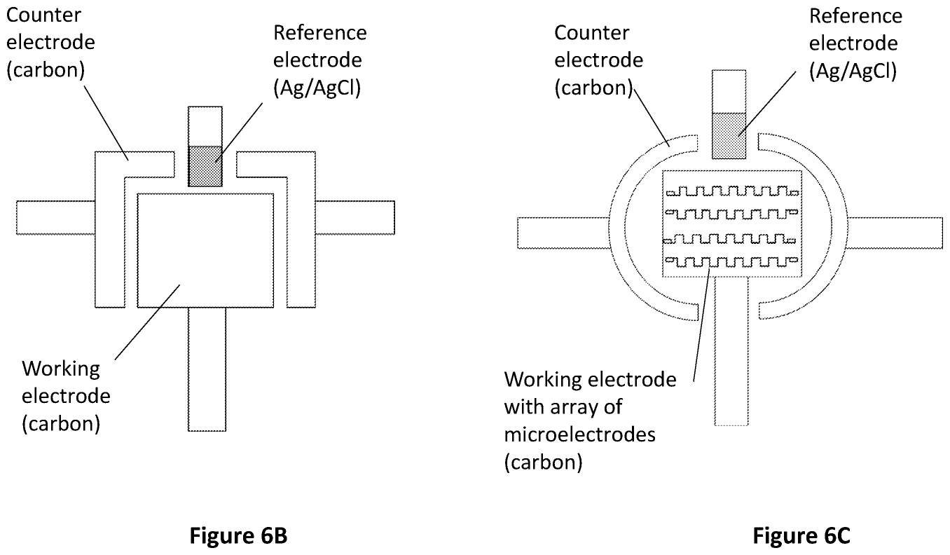

FIG. 6A shows an exemplary electrochemical sensor for detecting pathogenic/bacterial metabolites. The electrochemical sensor 600 has a working electrode 610, a reference electrode 620 and a counter electrode 630 provided on a substrate 605. The electrodes may be implemented as a set of screen-printed electrodes. For instance, the electrodes may be fabricated by printing different types of ink on the substrate 605. The substrate may be made of plastic or ceramic.

In this example the working electrode 610 has a circular profile or disc shape. The reference electrode 620 and the counter electrode 630 have a curved geometry surrounding the reference electrode 610. The working electrode 610 and the counter electrode 630 may be made using a carbon ink. For example, the reference electrode 610 may be made using a silver ink or a silver chloride ink. Depending on the application different types of inks may be used for the working electrode, including platinum, gold, palladium, and copper among others.

A set of connections pads 641, 642, 643 is provided to connect each electrode to a desired potential. For instance, the connections pads may be used with a potentiostat (not shown). The potentiostat may be used to maintain the potential of the working electrode 610 at a constant level with respect to the reference electrode 620 by adjusting a current at the counter electrode 630.

The zoom in view of the working electrode 610 shows the surface of the working electrode functionalized with cyclodextrin molecules. A monolayer of cyclodextrin molecules is attached at the surface of the working electrode. Each cyclodextrin has a cavity to receive a metabolite hence forming a cyclodextrin metabolite complex. In this example each cyclodextrin receives a phenazine molecule forming a cyclodextrin-phenazine complex. In most cases a single phenazine metabolite is expected to fit into the cyclodextrin cavity, however for small phenazine metabolites two molecules might fit into the cavity. It will be appreciated that this closeup view is only provided for illustrative purpose and is not a scaled representation. It will also be appreciated that the geometry of the electrodes may vary.

FIG. 6B shows a set of electrodes having another geometry. In this example the working electrode and the reference electrode have a square shape of different size. A pair L-shaped of counter electrodes are provided on the right and left side of the working electrode. The reference electrode is provided above the working electrode between the arms of the counter electrodes. A connection extends from the main body of each electrode.

FIG. 6C is a diagram of a set of electrodes having another geometry. The electrode geometry is similar to the configuration of FIG. 6B, however in this example the counter electrodes have a crescent shape or U shape surrounding the cyclodextrin modified screen printed working electrode. The working electrode is also provided with an array of microelectrodes. The microelectrodes can be used to enhance further the sensitivity of detection.

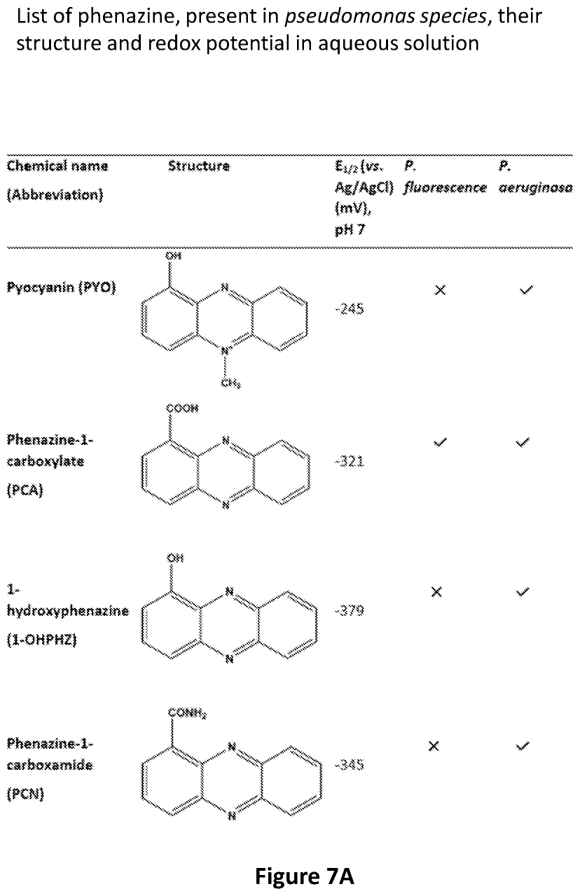

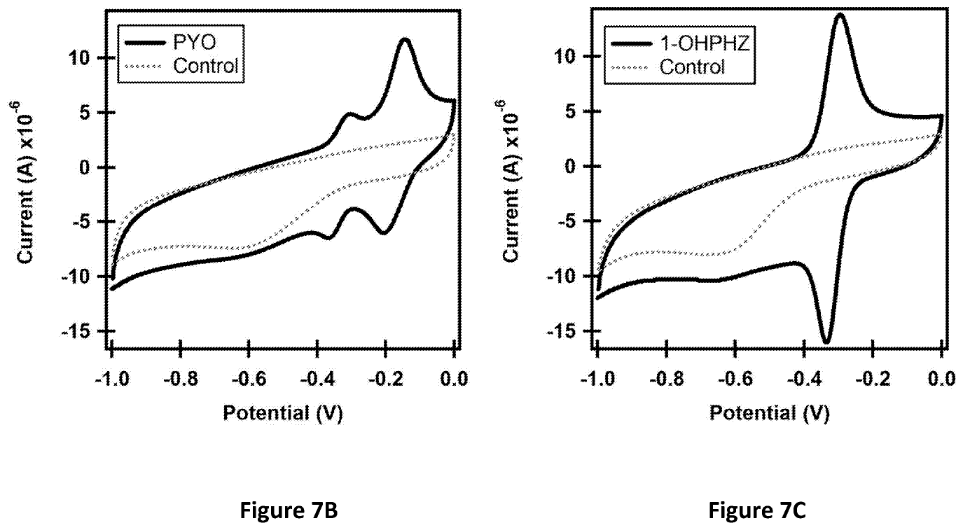

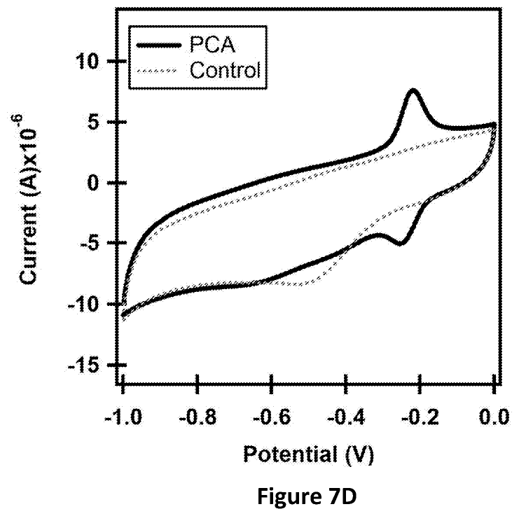

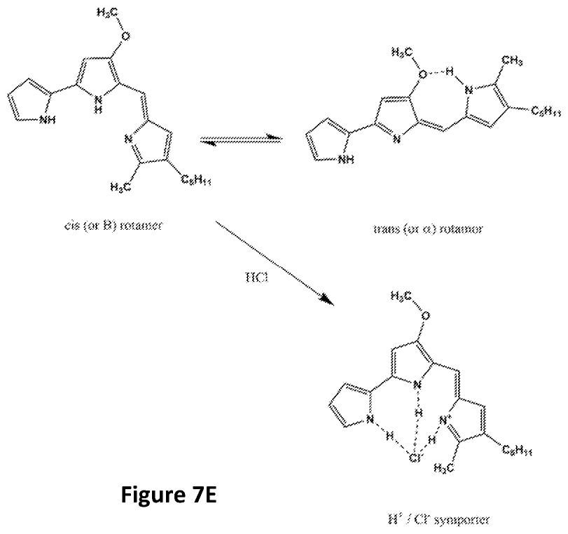

FIG. 7A shows a list of phenazine molecules present in Pseudomonas species (Pseudomonas phenazines), along with their structure and redox potential in aqueous solution. The list includes pyocyanin (PYO), phenazine 1 carboxylic acid (PCA), 1-hydroxyphenazine (1-OHPHZ), phenazine-1-carboxamide (PCN), and phenazine-1-carboxylic acid.

Pseudomonas fluorescens (P. fluorescens) and Pseudomonas aeruginosa (P. aeruginosa) are both biofilms forming bacterial species of the Pseudomonas genus. They are both gram-negative, rod-shaped, polar flagellated and aerobic. However, there is a key difference, P. aeruginosa is an opportunistic human pathogen which is virulent while the P. fluorescens is a plant growth promoting bacterium. Several bacterial species including P. fluorescens and P. aeruginosa, produce different variants of phenazines as secondary metabolites and quorum sensing molecules (Marco, Lluïsa Vilaplana & M.-Pilar, “Phenazines as potential biomarkers of Pseudomonas aeruginosa infections: synthesis regulation, pathogenesis and analytical methods for their detection,” Analytical and Bioanalytical Chemistry, vol. 412, p. 5897-5912, 2020). Both P. fluorescens and P. aeruginosa, have the operons for the production of phenazine 1 carboxylic acid (PCA) from chrorismates. However, only P. aeruginosa has diverse and specific enzymes required for the transformation of PCA to other phenazines such as phenazine-1-carboxamide (PCN), pyocyanin (5-N-methyl-1-hydroxyphenazine, PYO), 1-hydroxyphenazine (1-OH-PHZ). PYO production is associated with a high percentage of P. aeruginosa isolates and is considered to be the most potent virulence factor associated with the bacteria. These redox-active pigments control the redox status, gene expressions and metabolic flux and have been reported to influence antibiotic susceptibility. These phenazine metabolites have different redox potentials.

FIGS. 7B, 7C and 7D show the cyclic voltammetry graphs of β-CD/SPCE in 50 μM PYO, 1-OHPHZ and PCA in PBS, respectively. The doted lines represent the blank. The cyclic voltammetry plots of 50 μM PYO, PCN and 1-OHPHZ in phosphate buffer, show two redox peaks for PYO at around −0.144 V and −0.312 V. In contrast, only one well-defined peak for PCA and 1-OHPHZ was observed at −0.22 V and −0.296 V, respectively.

FIG. 7E is a diagram illustrating the structure of prodigiosin, a pigment from microbial source such as Serratia marcescens. Serratia marcescens is an opportunistic, gram-negative pathogen, which is widespread in the environment and can cause of hospital acquired infections such as urinary tract infections, respiratory tract infections and wound infections. Serratia species are capable of producing a pigment, prodigiosin, as a secondary metabolite. Prodigiosin production is dependent on ambient conditions such as media composition, temperature, and pH. Structurally, it contains three pyrrolic rings, with a pyrrolyl dipyrromethene skeleton and a 4-methoxy, 2-2 bi pyrrole ring system. The molecule has been extensively studies using spectroscopic methods. It can exist in two forms in solution as a mixture of cis (or β) and trans (or α) rotamers in a ratio that is dependent on the pH of the solution. In ethanol-water mixtures, Prodigiosin has a pKa value of 7.2.

The molecule is reported to show two peaks in the visible part of the spectrum with maxima at 537 nm and 470 nm (2.31 and 2.64 eV, respectively). The lower energy peak dominates at acidic pH and the higher energy one at basic pH. Prodigiosin production is commonly estimated spectrophotometrically using the Haddix and Werner methods.

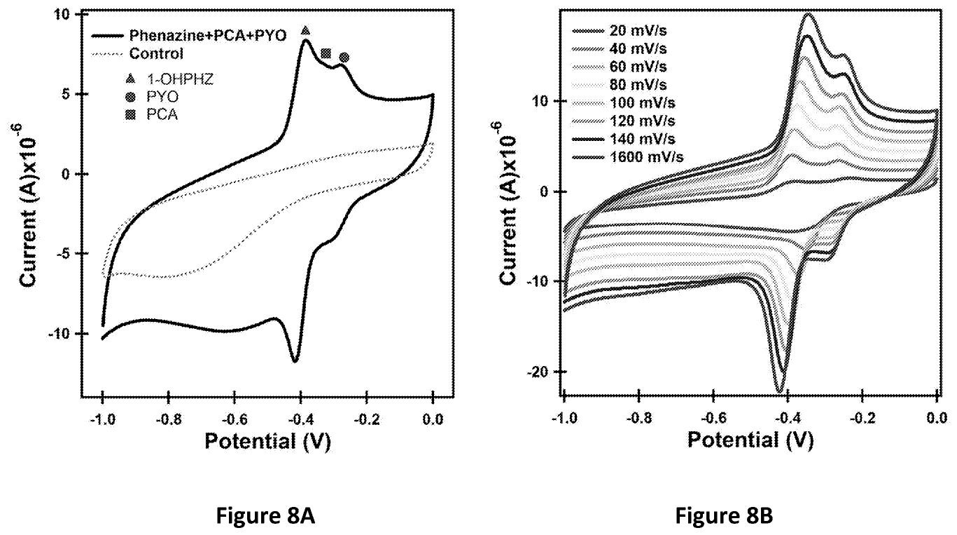

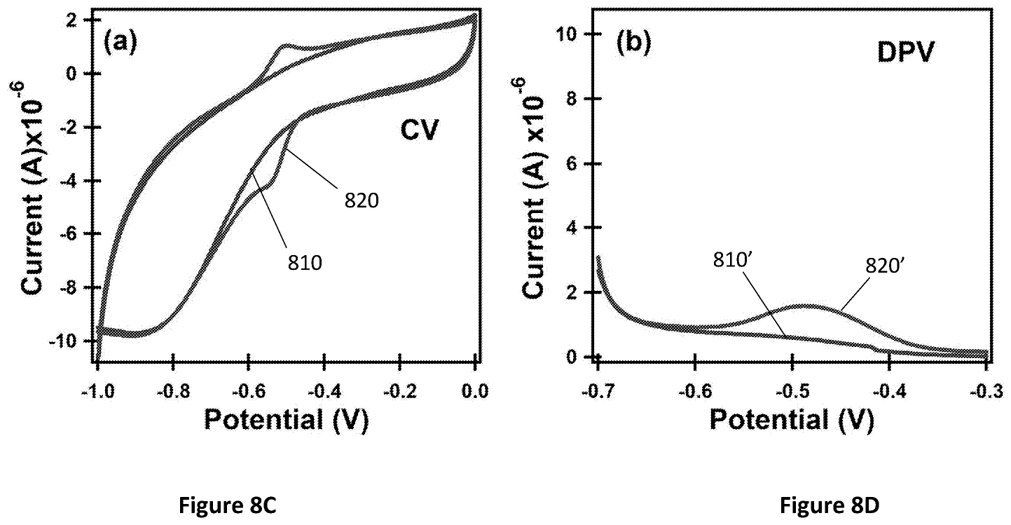

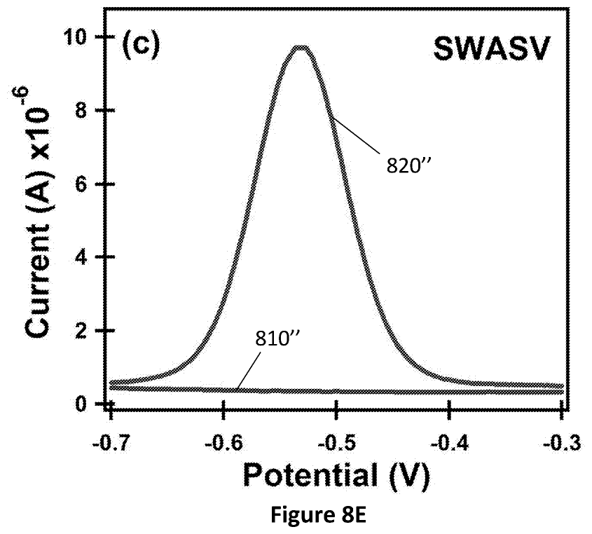

FIG. 8A shows the cyclic voltammetry (CV) graph of a β-CD modified screen printed carbon electrode (SPCE) in a ternary solution mixture of 1-OHPHZ, PCA and PYO in PBS pH 7 (solid line) and control (dot line). The mixture includes 12.5 μM 1-OHPHZ, 12.5 μM PYO and 12.5 μM PCA in PBS. The CV graph presents well-defined redox peaks for 1-OHPHZ and PYO at −0.384 V and −0.278 V respectively, whereas partially overlapped peak of the PCA was observed in the voltammograms. To get information on electrochemical reaction mechanism, the effect of scan rate on the peaks current and potential were evaluated for 12.5 μM ternary solution mixture of 1-OHPHZ, PCA and PYO in PBS (pH 7) at the β-CD/SPCE.

FIG. 8B shows the cyclic voltammetry graphs of β-CD/SPCE at different scan rate from 20 mV/s to 160 mV/s in 12.5 μM (1-OHPHZ+PYO+PCA). The anodic and cathodic peak currents increase with scan rate in the range 20 mV/s to 160 mV/s. The cathodic and anodic peaks currents increase linearly for all three phenazine compounds with the v as expected for the redox reaction of surface-confined molecules.

The electrochemical sensor as described above with reference to FIG. 6 may be used to perform various types of electrochemical techniques for the detection of pathogenic metabolites including phenazine and pigment molecules. These techniques may include voltammetry, amperometry or impedance techniques. Voltammetry techniques may be implemented in different ways, including linear sweep voltammetry, cycling voltammetry, stripping voltammetry, differential pulse voltammetry, among others. Among these various techniques stripping voltammetry and in particular square wave adsorptive stripping voltammetry (SWASV) has been identified has a preferred technique for the detection of phenazine metabolites in Pseudomonas species.

FIGS. 8C, 8D, and 8E show a cyclic voltammogram (CV), a different pulse voltammogram (DPV) and square wave adsorptive stripping voltammogram (SWASV) obtained before 810 and after 820 adding 3 μM 1-OHPHZ into PBS. SWASV was found to deliver faster electrochemical responses and showed a wide dynamic range and displayed in higher sensitivity compared to cyclic voltammetry and differential pulse voltammetry techniques. For instance SWASV shows peak current 5 times larger than differential pulse voltammetry.

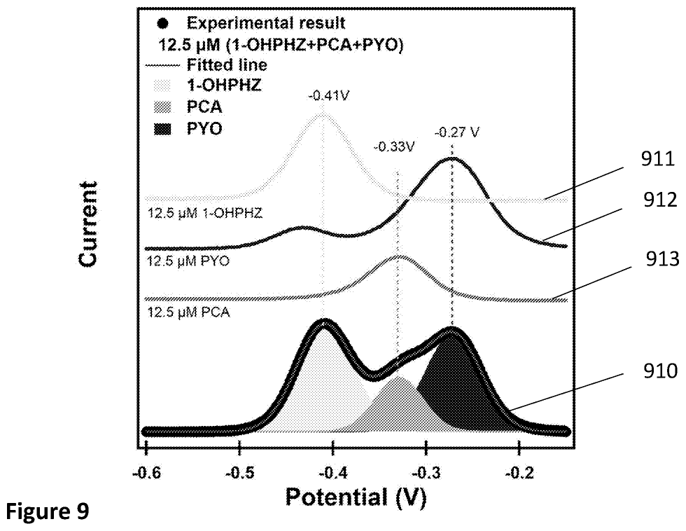

FIG. 9 shows a SWASV voltammogram (average of multiple measurements) obtained for a ternary solution mixture of 12.5 μM 1-OHPHZ, 12.5 μM PCA and 12.5 μM PYO in PBS buffer at β-CD/SPCE. The voltammogram 910 has several peaks that can be deconvoluted to the three peaks corresponding to OHPHZ, PCA and PYO individually, at −0.41V, −0.33V and −0.27V (vs. Ag), respectively (see curves 911,912 and 913).

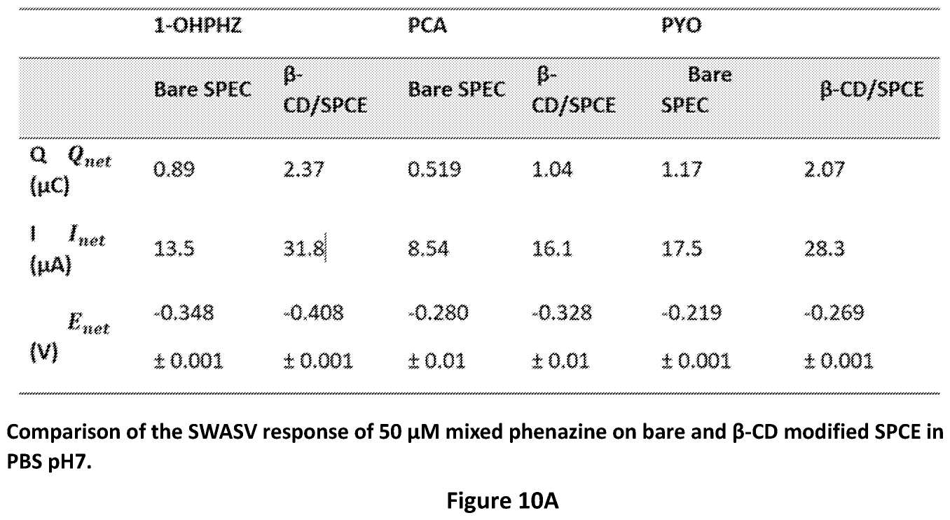

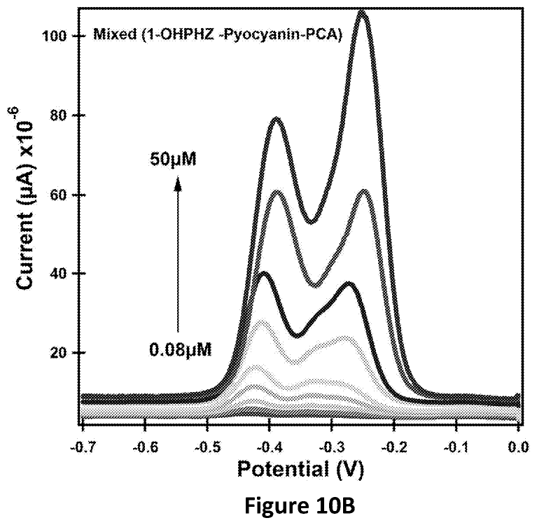

FIG. 10A is a table showing SWASV parameters (charge Qₙₑₜ, the current Iₙₑₜ and the potential Eₙₑₜ at the working electrode) obtained for the detection of a three phenazine compounds when using a bare electrode and a beta-cyclodextrin modified electrode (β-CD/SPEC), respectively. The comparison of SWASV measurements using the same sample solution on the bare SPCE and β-CD/SPCE, under the same experimental conditions, reveals the enhancement of signal obtained using β-CD/SPCE. The enhancement of analytical signal is due to the pre-concentration of the analytes entrapped in the cavity of the β-CD in proximity with the electrode surface.

The optimum conditions are assumed to be a phosphate buffer solution (pH7), accumulation time (t_{acc}) of 120 s, frequency (f) of 25 Hz, pulse amplitude (E_{sw}) of 25 mV and step potential (ΔE_{S}) of −0.8V. Under these conditions a series of voltammograms of increasing concentration phenazine from 0.78 μM-200 μM were recorded by SWASV on β-CD modified SPCE.

FIG. 10B is a diagram showing SWASV voltammograms obtained on β-CD modified SPCE for different mixed of phenazine concentration (from 0.08 μM to 50 μM) in PBS buffer.

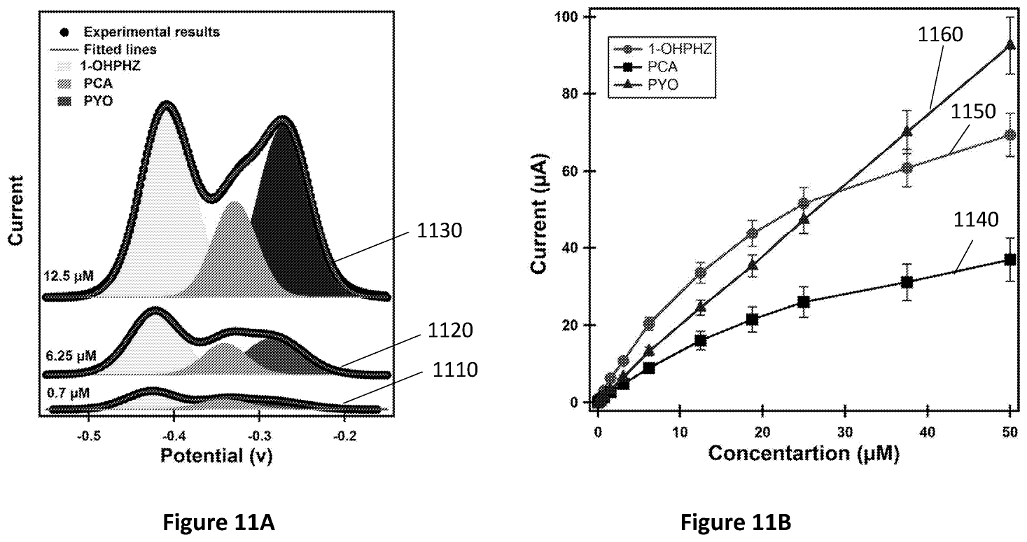

FIG. 11A is a diagram showing SWASV voltammograms obtained for different concentrations of mixed phenazines. The voltammograms 1110, 1120 and 1130 were obtained for a concentration of 0.7 μM, 6.25 μM and 12.5 μM of mixed phenazine, respectively.

FIG. 11B is a calibration chart showing the calibration curves (current versus concentration) for each phenazine molecule. The calibration curve 1140 is for PCA, the calibration curve 1150 is for 1-OHPHZ, and the calibration curve 1160 is for PYO.

The calibration curve 1160 is linear over the entire range spanning 0.08 μM-50 μM for PYO. The calibrations curves 1140 (PCA) and 1150 (1-OHPHZ) are linear between about 0.08 UM-2.5 μM.

The β-CD modified electrode exhibited enhanced sensitive detection for all three phenazine in comparison to the bare electrode. An increase in limit of detection LOD of was observed compared to the bare electrodes. The LOD (minimum concentration of phenazine) was different for the three different phenazines in the mixture: 1-OHPHZ<0.01 μM, PCA<0.05 μM, PYO<0.07 μM for modified electrode for n=6, (n is the number of data used in the calibration curve to calculate LOD).

This value is beneficial to biomedical application, sensitive detection of phenazine would enable the early detection of quorum sensing production of phenazine by colonising P. aeruginosa and P. fluorescence.

The electrochemical sensor of the disclosure can be used with bacterial cultures for detecting bacterial metabolites generated directly in a bacterial growth medium. In the following example the proposed method was applied for electrochemical detection of phenazine metabolites from Pseudomonas fluorescens and Pseudomonas aeruginosa in lysogeny broth (LB) growth media pH7. Pseudomonas fluorescens and Pseudomonas aeruginosa were allowed to grow on the β-CD modified SPCEs to detect phenazine metabolites. SWASV was conducted at different time points during the bacterial biofilm formation.

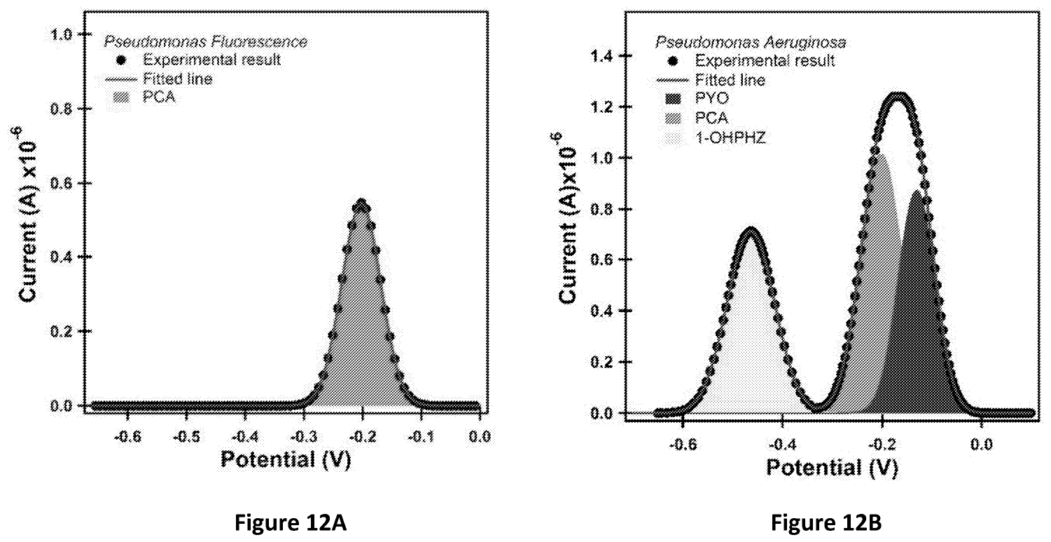

FIGS. 12A and 12B show the SWASVs of Pseudomonas fluorescence and Pseudomonas aeruginosa in LB media growth after 3 days (72 h), respectively. There is only one visible peak observed for Pseudomonas fluorescence at −0.20 V corresponding to the potential of PCA. According to the calibration curve, the estimated concentration of the PCA was calculated 0.41 μM after 3 days. However, Pseudomonas aeruginosa shows multiple peaks at −0.46 V, −0.20 V and −0.13 V. By comparison with the calibration curves, it can be concluded that the peaks are from 1-OHPHZ, PCA and PYO with the estimated concentration of 0.74 μM, 1.9 μM and 2.4 μM, respectively. Phenazine being pH-sensitive, it is possible that the pH was slightly basic thus shifting the potential. These results agreed well with the previous findings that Pseudomonas aeruginosa can secrete multiple phenazines, including pyocyanin, phenazine-1-carboxylate, phenazine-1-carboxamide and 1-OHPHZ.

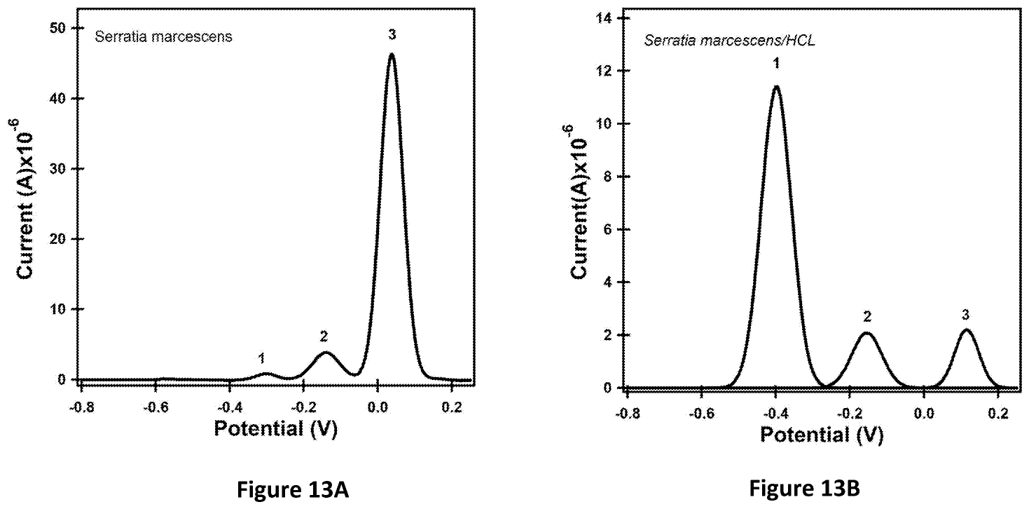

FIGS. 13A and 13B show the SWASVs of Serratia marcescens in LB medium growth after 3 days obtained in normal and acidic conditions (after adding HCL), respectively. In normal conditions (FIG. 13A) SWASVs of Serratia marcescens in LB medium after 3 days revealed three peaks (E¹=−0.30 V, E²=−0.14V, E³=0.03V vs. Ag pseudo reference electrode). The three peak currents intensities were

$$( {i}_{p}^{1} = 0.85 μA , {i}_{p}^{2} = 3.6 μA and {i}_{p}^{3} = 46 μA ) .$$

In acidic conditions (FIG. 13B) the peaks potential shifted to (E¹=−0.39V, E²=−0.15V, E³=0.11V vs. Ag pseudo reference electrode) with decrease in peak current intensities of

$$( {i}_{p}^{1} = 11.4 μA , {i}_{p}^{2} = 2 μA and {i}_{p}^{3} = 2.2 μA ) .$$

There are very few reports of electrochemical measurements of prodigiosin. Melvin and co-workers performed cyclic voltammetry measurements of the pure acetonitrile using a three-electrode cell consisting of a glassy carbon working electrode, a Pt spiral counter-electrode, and a silver wire pseudo reference electrode (Matt S Melvin, et al, “Influence of the a-ring on the redox and nuclease properties of the prodigiosins: importance of the bipyrrole moiety in oxidative DNA cleavage,” Chemical research in toxicology, vol. 15, pp. 742-8, 2002). They reported three peaks at (E¹=0.44V, E²=0.89 v and E³=1.54 V vs SCE with the second peak showing a shoulder at 1.06V) under acidic conditions to generate the protonated species a shift of E² (0.62 V) and a slight decrease in iₚ was reported. Although the measurements of FIG. 13 are not directly comparable, three distinct redox peaks are observed with an anodic shift in E³ accompanied by a decrease in current intensity. These results are consistent with the generation of the conjugate acid, which, as a positively charged species, is oxidized at a higher potential than the corresponding free base.

The results presented indicate that β-CD modified SPCE produced an improved resolution of the redox peaks produced by the quorum sensing molecules of biofilms response compared with unmodified screen-printed electrode. This effect is due to catalytic enhancement provided by the complexes formed between β-CD and electroactive quorum sensing molecules and/or metabolites (detectable molecules produced during biofilm formation). The proposed method can deconvolute redox peaks of metabolites from different bacterial species, thus offering a simple method for identifying and fingerprinting bacterial species.

The experiments presented above were conducted using the following chemicals and instruments: 1-OHPHZ (purity of 98%), Pyocyanin (purity of 98%), and β-cyclodextrin powder were purchased from Sigma-Aldrich. Phenazine-1-carboxylic acid was provided by Apollo Scientific. 1-OHPHZ and PYO stock solution (4.0×10⁻⁴ μM) were prepared in ethanol-phosphate buffer solution (1:10) as solvent. PCA stock solution (4.0×10⁻⁴ μM) was prepared in phosphate buffer solution. Electrochemical measurements were performed using a single screen-printed electrode (Micrux technologies, Gijón, Spain (S1PE)), consisting of a working electrode (carbon, diameter of 3 mm), carbon-based counter electrode and silver reference electrode. Cyclic voltammetry (CV) and square wave adsorptive stripping voltammetry (SWASV) measurements were performed with a Gamry PE-1000 potentiostat. The SWASV technique was applied under optimum conditions such as accumulation time (t_{acc}) of 120 s, frequency (f) of 25 Hz, pulse amplitude (E_{sw}) of 25 mV and step potential (ΔE_{S}) of −0.8V at β-CD modified screen-printed electrode. All measurements were made at pH 7.2 at ambient temperature. The surface morphology of the polymers was determined using scanning electron microscopy, Zeiss sigma 300 VP. For instance the surface may be checked to confirm that the cyclodextrin molecules form a monolayer.

For bacterial culture P. fluorescens, P. aeruginosa and S. marcescens has grown overnight at 32° C. with continuous shaking in the 10 ml of LB broth. Glucose (20 gr/L) was supplied as an electron donor.

The electrochemical sensor of the disclosure uses a cyclodextrin modified electrode to identify bacteria by their metabolites. The capability of differentiating bacterial species by the identification of specific metabolites that can act as a ‘fingerprint’ for the species and provides a powerful platform for monitoring bacterial activity. The cyclodextrin modified electrode permits to improve sensitivity. Adsorptive Stripping Voltammetry techniques was also found to deliver faster electrochemical responses and showed a wide dynamic range and displayed in higher sensitivity compared to other electrochemical techniques. The electrochemical sensor can be used to perform rapid and precise identification of infectious microorganisms across a range of applications where microbial contamination can cause serious issues ranging from microbial resistance to corrosion.

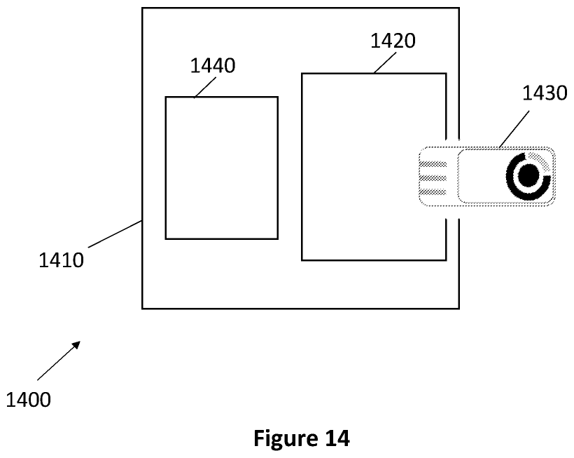

FIG. 14 is a diagram of a portable device. The portable device 1400 includes a housing 1410 provided with an electronic circuit 1420 and a slot for receiving a chip 1430. The chip 1430 includes a set of electrodes and connection pads for connection to the electronic circuit 1420. The chip 1430 may be implemented as the electrochemical sensor of FIG. 6A, or 6B or 6C. The chip 1430 may be a disposable chip. The electronic circuit 1420 is adapted to perform an electrochemical technique on the chip 1430. For instance, the electronic circuit may include a potentiostat and a processor for executing an algorithm performing data analysis. The electronic circuit 1420 is coupled to a display 1440 for communicating a result to a user of the device. The portable device may be used for a variety of applications. For instance, the chip 1430 may be designed for detecting bacterial contamination in water. In another application the chip 1430 may be designed for detecting bacterial contamination in milk etc. . . . .

It will be appreciated that the electrochemical sensor described in the present description could also be integrated as part of a bandage such as a skin patch, for instance to monitor bacterial levels on a wound.

A skilled person will therefore appreciate that variations of the disclosed methods and arrangements are possible without departing from the disclosure. Accordingly, the above description of the specific embodiments is made by way of example only and not for the purposes of limitation. It will be clear to the skilled person that minor modifications may be made without significant changes to the operation described.

1. An electrochemical sensor for detecting pathogenic metabolites, wherein the electrochemical sensor comprises a first electrode modified with oligosaccharide molecules.

2. The electrochemical sensor as claimed in claim 1, wherein the first electrode is modified with cyclic oligosaccharide molecules.

3. The electrochemical sensor as claimed in claim 2, wherein the cyclic oligosaccharide molecules comprise cyclodextrins or modified cyclodextrins or a combination of both.

4. The electrochemical sensor as claimed in claim 3, wherein the first electrode is modified with at least one of alpha-cyclodextrins, beta-cyclodextrins and gamma-cyclodextrins.

5. The electrochemical sensor as claimed in claim 1, wherein the first electrode is modified by electro-polymerization.

6. The electrochemical sensor as claimed in claim 1, comprising a second electrode and a third electrode, wherein the first electrode is a working electrode, the second electrode is a reference electrode, and the third electrode is a counter electrode.

7. The electrochemical sensor as claimed in claim 6, wherein the first electrode, the second electrode and the third electrode are a screen printed electrodes.

8. An electrochemical system comprising an electrochemical sensor as claimed in claim 1, and a potentiostat coupled to the electrochemical sensor, wherein the potentiostat is configured to perform an electrochemical technique.

9. The electrochemical system as claimed in claim 8, wherein the electrochemical sensor and the potentiostat are integrated in a portable device or a wearable device.

10. A method of detecting pathogenic metabolites, the method comprising
providing a first electrode modified with oligosaccharide molecules;
applying a sample on the first electrode; and
measuring an electrochemical response using the first electrode to detect pathogenic metabolites in the sample.

11. The method as claimed in claim 10, wherein the pathogenic metabolites comprise redox-active metabolites.

12. The method as claimed in claim 10, wherein the pathogenic metabolites comprise phenazine metabolites.

13. The method as claimed in claim 12, wherein the phenazine metabolites comprise at least one of pyocyanin (PYO), phenazine 1 carboxylic acid (PCA), 1-hydroxyphenazine (1-OHPHZ), and phenazine-1-carboxylic acid (PCN).

14. The method as claimed in claim 10, comprising performing an electrochemical technique to obtain the electrochemical response.

15. The method as claimed in claim 14, wherein the electrochemical technique is a stripping voltammetry technique, and the electrochemical response comprises a voltammogram.

16. The method as claimed in claim 15, wherein the stripping voltammetry technique is adsorptive stripping voltammetry.

17. The method as claimed in claim 10, wherein the first electrode is modified with cyclic oligosaccharide molecules.

18. The method as claimed in claim 10, further comprising determining an amount of pathogenic metabolites.

19. The method as claimed in claim 10, further comprising deconvoluting the electrochemical response to identify one or more redox peaks associated with a specific pathogenic species.

20. A method of manufacturing an electrochemical sensor for detecting pathogenic metabolites, the method comprising providing a first electrode and modifying the first electrode with oligosaccharide molecules.

21. The method as claimed in claim 20, wherein modifying the first electrode comprises performing electro polymerization.
